(19) United States
(12) Patent Application Publication
(10) Pub. No.: US 2026/0275212 A1
(43) Pub. Date: Sep. 17, 2026

(54) MARKING OF HYDROCARBON PRODUCTS

(71) Applicant: SICPA HOLDING SA, Prilly (CH)

(72) Inventors: Juan LOPEZ GEJO, Lausanne (CH); Ünige LASKAY, St. Sulpice (CH); Oscar DE FEO, Lausanne (CH)

(21) Appl. No.: 19/164,153

(22) PCT Filed: Mar. 6, 2024
(86) PCT No.: PCT/EP2024/055920

§ 371 Date: Sep. 10, 2025

(30) Foreign Application Priority Data
Mar. 10, 2023 (EP) 23161162.5

(51) Int. Cl. C10L 1/12; G01N 23/223

(52) U.S. Cl. CPC C10L 1/1208; C10L 2230/16; G01N 23/223

(57) ABSTRACT

The present invention relates to a method for marking a hydrocarbon product (HP) by using at least two markers, wherein the at least two markers have at least one same atom and/or functional group and the markers have different boiling points and/or different distillation profiles. The markers are selected in such a way that the entire range of the distillation temperature T(BpL) and T(BpE) of the HP is covered. An atom portion originating from individual markers M1 and M2 in a marker mixture Mₓ is determined by performing a regression analysis for at least part of the distillation temperature range of the HP. Based on said analysis a marker mixture Mₓ is calculated and added to mark a HP. The invention also relates to a fuel marked by the method and methods for detecting adulteration in HP.

### FIELD OF THE INVENTION

The present invention relates to the technical field of marking of hydrocarbon products (HP) and a method to mark HPs with at least two markers containing at least one same atom and/or functional group to ensure qualitative as well as quantitative determination of adulteration of the marked HP.

### BACKGROUND OF THE INVENTION

Marking of a product is described as the process of adding a security taggant or marker, which may be a molecule, an atom or a chemical compound, to a product that allows to track its origin or avoid its adulteration along the supply chain. Throughout the HP supply chain, various products such as crude oil, mineral oil, fuel oil, gasoline, diesel etc. may be subjected to markings to allow traceability of their origin or to avoid their adulteration with products of lower quality or with products submitted to lower or no tax. Many of these products may be exposed to a distillation process with the malicious intention of removing markers from the marked hydrocarbon product and thus eliminating the traceability.

For example, in many countries diesel is subsidized for certain industrial sectors such as agriculture or mining, while it is traded at a high level of taxation for the automotive sector. The result is a very significant price difference due to the different level of taxation which becomes an incentive for fraud i.e. sale of agricultural fuel as automotive fuel. To detect this type of fraud, governments mark agricultural fuel; if the marker is detected at the service stations selling automotive fuel, it can be proven that the fuel was being illegally sold. Criminals use distillation to remove the marker and then sell the product obtained free of the marker at the price of automotive fuel with the consequent impact on tax revenue.

In another example, in countries or regions, fuel is generally heavily subsidized and in some local distribution centers the selling price of the HP is significantly lower than the international market price. There is therefore a clear incentive to buy local subsidized fuel and export it illegally to further sell it at international prices with the economic benefit, which impacts the coffers of the producer country. In these cases, the local fuel is marked and controlled at the borders. A fraud or adulteration is detected if the exported loading shows the presence of the marker. The smuggling networks once again use distillation as a method of laundering the product. Even when the marker is not completely removed and the authorities manage to detect it in a suspicious shipment, the latency of the concentration due to the distillation process does not allow the authorities to make a fair quantification of how much product has been confiscated and therefore it is impossible to apply the current legislation which generally imposes penalties and fines depending on the damage caused to the public coffers.

Since most of the products present in the hydrocarbon distribution chain are complex mixtures of hydrocarbons, they all present a distillation curve or profile over a wide temperature range, which makes it impossible to be homogeneously measured by a single marker. As a result, through a distillation process the marker is distributed inhomogeneously in the different fractions, completely losing the original marker concentration and leaving many fractions completely free of marker. Or in the worst case, the marker undergoes complete degradation resulting in distillation products which are completely free of marker. Thus, qualitative as well as quantitative detection of marker in the marked product is important as it aids the prevention of illicit activities for monetary benefits.

Croud et. al. (Fuel Processing Technology, 144, (2016), 341-347) discloses a method for marking diesel with a single marker which is resistant to distillation. Thus, fraudulent attempts to separate the diesel from the marker are doomed as the marker may be detected by GC-MS in all distillation fractions of the marked diesel. However, this method suffers from the drawback of not permitting quantification of the adulteration unless complex and time-consuming calibration process are required.

U.S. Pat. No. 9,688,930 B2 discloses the use of several markers with different boiling points to cover the entire range of the marked hydrocarbon product. Hence, fraudulent distillation attempts are detected as all distillation fractions will comprise at least one of the markers. However, this method suffers from the drawback that quantification of the adulteration may be performed only through complex and time-consuming calibration and validation processes.

U.S. Pat. No. 10,808,192 B2 discloses the use of two markers: one with a low boiling point (A) and one with a high boiling point (B). The sample is labeled with a given A/B ratio. A fractionation through distillation of the marked hydrocarbon product is performed to determine the A/B ratio in each distillation fraction. When an unknown sample is analyzed, the concentration of A and B is measured, and the A/B ratio may be calculated and correlated with the previously determined ratios. However, this method suffers from the drawback that no correlation with the previously determined ratios may be performed in case only one of the marker A or B is detected and measured in the adulterated product.

Thus, there is a need to provide a suitable, convenient and robust method of marking a hydrocarbon product, which not only uncovers fraudulent adulteration attempts, particularly through distillation and/or dilution, but also allows easy marker detection as well as its quantification.

### SUMMARY OF THE INVENTION

In a first aspect the invention relates to a method for marking a hydrocarbon product (HP) comprising the steps of:
A. Selecting a HP to be marked and recording the distillation profile of HP to determine the starting temperature T(BpL) and the ending temperature T(BpE) at which distillation occurs;
B. Selecting at least two compatible and distillable markers (M1, M2) for marking the HP, said markers comprising at least one same atom and/or functional group to be analyzed by a suitable analytical technique, wherein M1 and M2 individually have different boiling points and/or different distillation profiles,
   wherein one of M1 or M2 has a boiling point temperature T(BpM1) being lower than T(BpL) or one of M1 or M2 has a boiling point temperature being within the distillation temperature range of HP [T(BpL)<T(BpM1)<T(BpE)] and being at most 20% higher than T(BpL), and
   wherein one of M1 or M2 has a boiling point temperature T(BpM2) higher than T(BpE) or the other of M1 or M2 has a boiling point temperature being within the distillation temperature range of HP [T(BpL)<T(BpM2)<T(BpE)], and being at most 20% lower than T(BpE);
C. Selecting a total atom concentration Cₜ-value, wherein Cₜ-value is selected based on the analytical technique and the at least one same atom and/or functional group to be analyzed by said analytical technique, selected in step B);
D. Preparing individual solutions n₁ and n₂ of markers M1 and M2 in the HP, respectively, at preferably the Cₜ-value, and determining before fractional distillation the initial concentration and the initial total mass of the at least one same atom and/or functional group in n₁ and n₂ by the analytical technique selected in step B);
E. performing separately fractional distillation of the solutions n₁ and n₂ to obtain fractions F;
F. determining the concentration and the total mass of the at least one same atom and/or functional group in each fraction F by the analytical technique selected in step B);
G. calculating the atom mass amount percentage (%) relative to the initial total mass of the at least one same atom and/or functional group determined in step D) in each fraction F and determining the cumulative atom mass amount percentage for each fraction F;
H. determining an atom portion originating from individual markers M1 and M2 in a marker mixture Mₓ by performing a regression analysis to determine a relationship between the cumulative atom mass amount percentage for each fraction F as determined in step G) for each of the solutions n₁ and n₂ and the distillation profile of HP determined in step A) for at least part of the distillation temperature range of HP;
I. calculating a marker mixture Mₓ composition based on the atom portion determined in step H) for each marker M1 and M2, the weight fraction of each atom and/or functional group in each marker M1 and M2 and the purity of markers M1 and M2;
J. preparing the marker mixture Mₓ by mixing the marker amounts determined in step I) and adding it to the hydrocarbon product HP at about the Cₜ-value.

In a second aspect the invention relates to a fuel marked by the method described herein.

In a another aspect the invention relates to a method of detecting adulteration in a hydrocardon product (HP) comprising the steps of:
a) sampling a HP marked according to the method described herein, said HP having a predetermined Cₜ-value;
b) detecting and calculating the Cₜ-value in the sampled HP by a suitable analytical technique;
c) assessing the potential adulteration of the HP by comparing the Cₜ-value determined in step b) with the pre-determined Cₜ-value of step a).

In a final aspect the invention relates to a method of detecting adulteration in a hydrocarbon product (HP) comprising the steps of:
a) sampling a first HP;
b) detecting and, if present, quantifying an atom and/or functional group by a suitable analytical technique;
c) determining the amount of a second HP marked according to the method described herein, based on the quantified atom and/or functional group from step b).

### BRIEF DESCRIPTION OF DRAWINGS/FIGURES

FIG. 1a-c illustrate the distillation curves of example E1, in particular the distillation profile of the neat hydrocarbon product HP1 (gasoline) (FIG. 1a), the distillation profiles of the three solutions of the three markers E1-M1, E1-M2 or E1-M3, each dissolved in HP1 (FIG. 1b), and the distillation profiles of the neat hydrocarbon product HP1 and the distillation profile of a marker mixture Mₓ including fractions of E1-M1, E1-M2 and E1-M3 in HP1 (FIG. 1c) as calculated by regression analysis.

FIG. 2a-c illustrate the distillation curves of example E2, in particular the distillation profile of the neat hydrocarbon product HP2 (diesel) (FIG. 2a), the distillation profiles of four solutions of the four markers E2-M1, E2-M2, E2-M3 or E2-M4, each dissolved in HP2 (FIG. 2b), and the distillation profiles of the neat hydrocarbon product HP2 and the distillation profile of a marker mixture Mₓ including fractions of E2-M1, E2-M2, E2-M3 and E2-M4 in HP2 (FIG. 2c) as calculated by regression analysis.

FIG. 3a-c illustrate the distillation curves of example E3, in particular the distillation profile of the neat hydrocarbon product HP3 (kerosene) (FIG. 3a), the distillation profiles of the three solutions of the three markers E3-M1, E3-M2 or E3-M3, each dissolved in HP3 (FIG. 3b), and the distillation profiles of the neat hydrocarbon product HP3 and the distillation profile of a marker mixture Mₓ including fractions of E3-M1, E3-M2 and E3-M3 in HP3 (FIG. 3c) as calculated by regression analysis.

### DETAILED DESCRIPTION

### Definitions

The following definitions are to be used to interpret the meaning of the terms discussed in the description and recited in the claims.

As used herein, the article “a” indicates one as well as more than one and does not necessarily limit its referent noun to the singular.

As used herein, the term “about” means that the amount or value in question may be the value designated or some other value about the same. The phrases are intended to convey that similar values within a range of ±5% of the indicated value promote equivalent results or effects according to the invention.

As used herein, the term “at least one” is meant to define one or more than one, for example one or two or three.

As used herein, the term “and/or” means that either all or only one of the elements of said group may be present. For example, “A and/or B” shall mean “only A, or only B, or both A and B”. In the case of “only A”, the term also covers the possibility that B is absent, i.e. “only A, but not B”.

The term “comprising” as used herein is intended to be non-exclusive and open-ended. The term “comprising” also covers, as a particular embodiment thereof, the more restrictive meanings of “consisting essentially of” and “consisting of”.

Where the present description refers to “preferred” embodiments/features, combinations of these “preferred” embodiments/features shall also be deemed as disclosed as long as this combination of “preferred” embodiments/features is technically meaningful.

“Atom” means an element (from the periodical table) being detectable in a quantitative manner by a selected analytical technique.

“Functional group” means a chemical moiety being detectable in a quantitative manner by a selected analytical technique. A functional group contains at least two different atoms.

“Marker” means a chemical compound containing at least one atom and/or functional group.

“Distillation profile” is the graphical representation obtained by plotting the amount in percent of the HP distilled at a specific temperature, as depicted in the figures.

“T(BpL)” is the starting temperature at which the first drops of the distillate are obtained, when performing the distillation of a HP.

“T(BpE)” is the end temperature at which the final drops of the distillate are obtained, when performing the distillation of a HP.

“Distillate” is the liquid that results from the condensing of vapour produced by heating a to be distilled liquid. It is the product recovered by distillation when heated from T(BpL) to T(BpE).

“Mₓ” is the marker mixture obtained by mixing at least two markers M1 and M2, wherein the amounts of the individual markers are determined based on the method described herein.

The method described herein is primarily, but not exclusively, aimed to the marking of a hydrocarbon product (HP). The term HP refers to any liquid hydrocarbon including but limited to either refined or unrefined petroleum products such as crude oil, naphtha, gasoline, diesel fuel, jet fuel, kerosene, propane, lubricant (e.g. engine oil), hydraulic fluid, natural gas. It is possible to also employ the method described herein to mark other distillable products.

The method of marking described herein comprises the steps A)-J). In a preferred embodiment, the steps are to be performed one after the other as defined. However, it is well within a skilled person's expertise to rearrange some of the steps or perform them in parallel without losing the essence of the method and where it is logical to do so. As an example, steps A)-C) maybe be performed in another order, without affecting the eventual result.

The method of marking described herein involves step A), wherein the HP to be marked is subjected to distillation to record the distillation profile of the HP. The distillation profile aids to determine among others the T(BpL) and the T(BpE) temperatures of the distilled HP. The distillation maybe performed by any standard distillation apparatus suitable for the purpose, preferably using the ASTM D-86 norm.

Step B), which may be performed after step A), comprises selecting at least two compatible and distillable markers (M1, M2) for marking the HP, said markers comprising at least one same or identical atom and/or functional group to be analyzed by a suitable analytical technique, wherein M1 and M2 individually have different boiling points and/or different distillation profiles. In a preferred embodiment, more than two markers are selected and even more preferably four markers M1-M4 are selected for marking the HP. It is also possible to have more than four i.e. “i” markers containing at least one same atom and/or functional group.

The at least two markers containing the at least one same atom and/or functional group are selected such that one of M1 or M2 has a boiling point temperature T(BpM1) being lower than T(BpL) or one of M1 or M2 has a boiling point temperature being within the distillation temperature range of HP [T(BpL)<T(BpM1)<T(BpE)] and being at most 20%, preferably 15%, higher than T(BpL), and wherein one of M1 or M2 has a boiling point temperature T(BpM2) higher than T(BpE) or the other of M1 or M2 has a boiling point temperature being within the distillation temperature range of HP [T(BpL)<T(BpM2)<T(BpE)], and being at most 20%, preferably 15%, lower than T(BpE). The selection of the at least two markers in such a manner ensures that the distillation range of the HP is covered by at least one marker contributing the at least one same atom and/or functional group to be analyzed and quantified.

The at least two markers described herein should be compatible and distillable with the to be marked HP. Compatibility means that the markers are selected which are stable, miscible in and compatible with the HP which is to be marked. For example, the markers satisfy certain requirements such as being environmental-friendly (i.e., not harmful to the air, water, soil components, living organisms, and the like), non-corrosive, non-volatile, non-toxic, non-reactive with each other or the HP. Furthermore, the markers are capable of being detected and quantified within the quantification range of the hydrocarbon product. Therefore, the nature of the markers and the amount added to the product must enable the subsequent detection of the at least one same atom and/or functional group contained in the markers by a suitable analytical technique. The quantity of each marker added is preferably calculated to provide the concentration of the at least one same atom and/or functional group greater than the lowest limit of quantification (LLQ). The LLQ may be the concentration at which a given minimum level of certainty can be guaranteed in the measurement of the concentration of the at least one same atom and/or functional group by the selected analytical technique.

The method of detection or analysis used may be selected from those which have the required sensitivity and selectivity according to the type of atom and/or functional group to be detected and quantified. Optical methods such as fluorescence, (Surface Enhanced) Raman spectroscopy, Fourier transform-infrared (FT-IR) or UV spectroscopy may be used in some cases. The at least one atom and/or functional group may be detectable by mass spectrometry or by atomic absorption technique employing elemental analysis. Suitable methods may be based on mass spectrometry such as inductively coupled plasma mass spectrometry (ICP-MS) or on emission spectroscopy such as ICP-optical emission spectroscopy (ICP-OES). A particularly advantageous method is based on X-ray fluorescence (XRF) spectroscopy. Another preferred method is the use of FT-IR to detect and quantify the amount of a functional group.

The at least two markers described herein contain at least one same or identical atom and/or functional group that may be analyzed and quantified by a suitable analytical technique. For example, if two different markers are selected, then they contain at least one same atom G and/or at least one same functional group H. In another embodiment, the at least two markers may contain another additional common analyzable atom and/or functional group.

According to one example the marker can be based on an alkane, whose formula is CₙH₂ₙ₊₂, where n=1,2,3 . . . . At least one hydrogen atom is substituted by an element which can be detected preferably by an XRF-analyzer. The resultant compound is having general formula CₙH₂ₙ₊₂₋ₘXₘ, where n=1,2,3 . . . , and m=1,2,3 . . . “X” is any element which can be detected preferably by an X-ray fluorescence analyzer. Simple example for this element is lithium (Li), an alkali metal, which forms one covalent bond with a carbon atom. In a preferred embodiment, the element “X” is a halogen.

Accordingly, the marker can be a halogenic compound, such as an alkyl halide having the general formula CₙH₂ₙ₊₂₋ₘXₘ where n=1,2,3 . . . , m=1,2,3 . . . “X” is a halogen such as fluorine (F), chlorine (Cl), bromine (Br), and iodine (I). Following are the chemical names and molecular formulae of some of suitable examples of the marker in the form of alkyl halide: 1,1,2,2-tetrachloroethane (C₂H₂Cl₄), 1,1,2-trichloroethane (C₂H₃Cl₃), pentachloroethane (C₂HCl₅), hexachloroethane (C₂Cl₆), 1,2,4-trichlorobenzene (C₆H₉Cl₃), 1,2,4,5-tetrachlorobenzene (C₆H₈Cl₄), ethyliodide (C₂H₅I), ethylbromide (C₂H₅Br), dichloro-1,2-dibromoethane (C₂H₂Cl₂Br₂), dichlorotribromoethane (C₂HCl₂Br₃), difluoro-1-chloroethane (C₂H₃F₂Cl), difluoro-1,2-dibromoethane (C₂H₂F₂Br₂), trifluoro-1,2,2-dibromoethane (C₂HF₃Br₂), tribromopropane (C₅H₇Br₃), dibromobenzene (C₆H₁₀Br₂), dibromoethane (C₂H₄Br₂), n-propylbromide (C₃H₇Br), parabromofluorobenzene (C₆H₄FBr), butylbromide (C₄H₉Br) and octylbromide (C₈H₁₅Br), 2-chloropropane (C₃H₇Cl), 1-chloropropane (C₃H₇Cl), chlorohexane (C₆H₁₃Cl), iodobenzene (C₆H₅l), 1,4-diiodobutane (C₄HsI₂), 1,6-diiodohexane (C₄H₈I₂), 1-iodohexadecane (C₁₆H₃₃I).

According to another example, the marker can be an organometallic or a halogenic compound in which at least one metallic element or at least one halogen, bonds with at least one carbon atom of an alkene (olefin), having the general formula (CₙH₂ₙ₋ₘ)Xₘ where n=2,3 . . . , m=1,2,3 . . . “X” is either an alkali metal or a halogen. An example of such a compound is bromoethylene having the molecular formula C₂H₃Br.

According to a further example the marker can be any of the above-mentioned compounds wherein silicon (Si), germanium (Ge), and the like, substitutes an atom of carbon. For example, diethyl silane (i.e., C₄H₁₂Si) is such a compound. It will be noted that silicon is detectable by X-ray fluorescence analyzer. Accordingly, “X” elements (i.e. halogens) do not need to appear in the compound, if the silicon, germanium and the like serve as the marking element detectable by the X-ray fluorescence analyzer. For alkanes, the general formula of the compound is Cₙ₋ₘH₂ₙ₊₂Yₘ, where n=1,2,3 . . . , m=1,2,3 . . . , m<n and where “Y” designates the silicon, germanium and the like. For alkenes (olefins), the general formula of the compound is (Cₙ₋ₘH₂ₙ₋ₘ)Yₘ, where n=1,2,3 . . . , m=1,2,3 . . . and where “Y” designates the silicon, germanium and the like.

Another class of particularly suitable markers are alkoxysilanes and alkyl alkoxysilanes such as methyltrimethoxysilane (CAS 1185-55-3), dimethoxydimethylsilane (CAS 1112-39-6), methoxytrimethylsilane (CAS 1825-61-2), ethoxytrimethylsilane (CAS 1825-62-3), triethoxymethylsilane (CAS 2031-67-6), isopropoxytrimethylsilane (CAS 1825-64-5), triethoxymethylsilane (CAS 2031-67-6), trimethoxy (octyl) silane (CAS trimethoxy (octadecyl) silane 3069-40-7), (CAS 3069-42-9), hexadecyltrimethoxysilane (CAS 16415-12-6), triethoxy (octyl) silane (CAS 2943-75-1), tetraethyl orthosilicate (CAS 78-10-4), dicyclopentyldimethoxysilane (CAS 126990-35-0), hexamethyldisiloxane (CAS 107-46-0), (3-aminopropyl) trimethoxysilane (CAS 13822-56-5), (3-aminopropyl)triethoxysilane (CAS 919-30-2), (N,N-dimethylaminopropyl) trimethoxysilane (CAS 2530-86-1), (3-mercaptopropyl) trimethoxysilane (CAS 4420-74-0), (3-mercaptopropyl)triethoxysilane (CAS 14814-09-6), triethoxy-3-(2-imidazolin-1-yl) propylsilane (CAS 58068-97-6) or cyclomethicones, which are a group of methyl siloxanes having a backbone of [(CH₃)₂SiO]ₙ. Suitable non-limiting examples include octamethylcyclotetrasiloxane, decamethylcyclopentasiloxane, dodecamethylcyclohexasiloxane, hexamethylcyclotrisiloxane, tetradecamethylcycloheptasiloxane (CAS 107-50-6), hexadecamethylcyclooctasiloxane (CAS 556-68-3), octadecamethylcyclononasiloxane (CAS 556-71-8), eicosamethylcyclodecasiloxane (CAS 18772-36-6), docosamethylcycloundecasiloxane (CAS 18766-38-6), tetracosamethylcyclododecasiloxane (CAS 18919-94-3), hexacosamethylcyclotridecasiloxane (CAS) or silanol compounds such as trimethylsilanol (CAS 1066-40-6) or compounds of the family of the silazanes such as but not limited to hexamethyldisilazane (CAS 999-97-3), 1,1,3,3-tetramethyldisilazane (CAS 15933-59-2) 1,3-diethyl-1,1,3,3-tetramethyldisilazane (CAS 17882-94-9), 2,2,4,4,6,6-hexamethylcyclotrisilazane (CAS 1009-93-4) or halogenated silanes such as but not limited to chlorodimethylsilane (CAS 1066-35-9), dichloromethylsilane (CAS 75-54-7), dichlorodimethylsilane (CAS 75-78-5), chloromethyltrimethylsilane (CAS 2344-80-1), bromotrimethylsilane (CAS 2857-97-8), iodomethyl)trimethylsilane (CAS 4206-67-1), chloro(chloromethyl)dimethylsilane (CAS 1719-57-9), trichloro(octadecyl) silane (CAS trichlorododecylsilane 112-04-9), (CAS 4484-72-4), trichloro(octyl) silane (CAS 5283-66-9), trichloro(phenethyl) silane (CAS 940-41-0), trichloro(hexyl) silane (CAS 928-65-4), methyltrichlorosilane (CAS 75-79-6), chloro(3-chloropropyl)dimethylsilane (CAS 10605-40-0), (3-chloropropyl) trimethoxysilane (CAS 2530-87-2), (3-bromopropyl) trimethoxysilane (CAS 51826-90-5), (3-iodopropyl) trimethoxysilane (CAS 14867-28-8), trimethylsilyl trifluoromethanesulfonate (CAS 27607-77-8).

If (FT-) infrared spectroscopy or (Surface Enhanced) Raman spectroscopy is selected as the analytical technique, suitable detectable and quantifiable functional groups may be used as part of the method described herein. Some examples are ketones and mixtures thereof such as but not limited to octanone (CAS 106-68-3), 2-nonanone (CAS 821-55-6), 2-decanone (CAS 693-54-9), 3-hexadecanone (CAS 18787-64-9), 5-octadecanone (CAS 18276-99-8) or hydrazines and mixtures thereof such as but not limited to 1,2-dimethyl-hydrazine (CAS 540-73-8), 1,2-diethyl-hydrazine (CAS 1615-80-1), 1,2-dipropylhydrazine (CAS 1615-83-4), 1,2-bis(1-methylethyl) hydrazine (CAS 3711-34-0), 1,2-dibutylhydrazine (CAS 1744-71-4), N,N-dimethylhydrazine (CAS 57-14-7), 1-aminopiperidine (CAS 2213-43-6), 1-amino-4-methylpiperazine (CAS 6928-85-4) or nitro-compounds and mixtures thereof such as but not limited to 1-nitroethane (CAS 79-24-3), 1-nitropropane (CAS 108-03-2), 1-nitrobutane (CAS 627-05-4), nitrobenzene (CAS 98-95-3) or cyclic esters and mixtures thereof such as but not limited to δ-undecalactone (CAS 710-04-3), γ-undecalactone (CAS 104-67-6), δ-dodecalactone (CAS713-95-1), δ-tridecalactone (CAS 7370-92-5), σ-tetradecalactone (CAS 2721-22-4) or ethers and mixtures thereof such as but not limited to dimethoxymethane (CAS), diethyl ether (CAS 60-29-7), diisopropyl ether (CAS 108-20-3), di-tert-butyl ether (CAS 6163-66-2), dibutyl ether (CAS 142-96-1), dihexyl ether (CAS 112-58-3), tert-butyl methyl ether (CAS 1634-04-4), 1-methoxyhexane (CAS 4747-07-3), formaldehyde diethyl acetal (CAS 462-95-3).

Step C) comprises the selection of a total atom concentration Cₜ-value, wherein the Cₜ-value is selected based on the analytical technique and the at least one same atom and/or functional group to be analyzed by said analytical technique, selected in step B). The Cₜ-value may vary from about 10 ppm up to about 2000 ppm, preferably from about 10 ppm to about 1000 ppm. The selection of the Cₜ-value is to be made such that reliable quantification of the at least one same atom and/or functional group can be achieved by the selected analytical technique.

To determine the individual distillation profile for the at least two markers M1 and M2, step D) comprises preparing individual solutions n₁ and n₂, respectively, in the HP which is to be marked. The concentration of the at least one same atom and/or functional group may be freely selected, however it is preferred to be close to the Cₜ-value itself and may even be less than the Cₜ-value. In a preferred embodiment, the concentration of the at least one same atom and/or functional group is selected to be equivalent to the Cₜ-value. Before performing fractional distillation of the solutions n₁ and n₂ individually, the initial concentration and initial mass of the at least one same atom and/or functional group is determined by the analytical technique selected in step B).

Step E) involves separately performing the fractional distillation of the solutions n₁ and n₂ to obtain fractions F1-X and F2-X, respectively. As an example, if 10 fractions are collected for solution n₁, these are denoted as F1-1 to F1-10. The distillation maybe performed by any standard distillation apparatus suitable for the purpose, preferably using the ASTM D-86 standard.

The concentration and total mass of the at least one same atom and/or functional group in each fraction F1-X is determined by the analytical technique selected in previous step B). The same operation is performed on the fractions F2-X of solution n₂ and for any additional marker My in a corresponding solution n_{y} prepared as described herein.

Step G) comprises calculating the atom mass amount percentage (%) relative to the initial total mass of the at least one same atom and/or functional group determined in step D) for each fraction F-X. As shown in table 3, column 4, when the initial atom mass is 1.11 mg (as detected in the undistilled sample n₁, step D) and fraction F1-1 contains 0.073 mg of the detected and quantified atom and/or functional group, the atom mass percentage (%) in fraction F1-1 can be calculated as 0.073/1.11*100=6.58%. Similarly, the remaining fractions may be calculated to determine the atom mass amount percentage (%) values.

Step G) further involves the calculation of the cumulative atom mass amount percentage (%) for each fraction F. Thus, as shown in table 3, last column, the cumulative atom mass amount percentage (%) is the total atom mass amount percentage (%) in each fraction F. It is clear to a skilled person that for the first fraction the cumulative atom mass amount percentage (%) will be equal to the total atom mass amount percentage (%). However, fraction F1-2 would contain a total atom mass percentage (%) originating from the previous fraction F1-1 as well as F1-2. Similarly, fraction F1-3 would contain the cumulative atom mass amount percentage originating from fractions F1-3 and the previous fraction F1-2. Thus, the cumulative atom mass amount percentage (%) value can be determined for all fractions in this manner. It should be noted that due to unavoidable errors when performing fractional distillation (e.g. temperature regulation, fraction collection mechanism), it is possible that the cumulative atom mass amount percentage (%) may deviate from the ideal value of 100%. However, this deviation does not affect the eventual analysis in step H).

Step H) comprises determining an atom portion (for example a percentage) originating from individual markers M1 and M2 in a marker mixture Mₓ by performing a regression analysis to determine a relationship between the cumulative atom mass amount percentage for each fraction F as determined in step G) for each of the solutions n₁ and n₂ and the distillation profile of HP determined in step A) for at least part of the distillation temperature range of HP. In particular, the regression analysis allows fitting a weighted sum of the cumulative atom mass amount percentage for each fraction F as determined in step G) for each of the solutions n₁ and n₂ to the distillation profile of HP. In other words, if the cumulative atom mass amount percentage of each marker M1 and M2 respectively has a weight coefficient b1 and b2, the fitting of the regression analysis allows determining the coefficients b1 and b2 such that a sum of b1 multiplied by the cumulative atom mass amount percentage for the solution n₁ and of b2 multiplied by the cumulative atom mass amount percentage for the solution n₂ comes as close as possible to the distillation profile of HP. The regression analysis allows determining the weights b1 and b2 of the weighted sum. The regression analysis preferably allows reducing a difference between the distillation profile of HP and the weighted sum of the cumulative atom mass amount percentage for each fraction F as determined in step G) for each of the solutions n₁ and n₂. The regression analysis can be any mathematical tool allowing for such a fitting and can in particular include a calculation of an average absolute deviation, a maximum absolute deviation, a mean worst case scenario absolute deviation, and/or of a least square median. The regression analysis may include any convex programming in which a cost function is well defined (which is for example the case of least square analysis). The regression analysis may be a least square optimization analysis, preferably a least square convex constrained optimization analysis. The fitting (which can also be referred to as a difference minimization) can be performed over the entire distillation temperature range of HP (referred to as “integral minimization”) or over sections thereof (referred to as “partial minimization”). Preferably, distinct partial minimizations are performed for different temperature sections such as to jointly cover the entire distillation temperature range of HP. This latter example allows a better fitting of each temperature section and is particularly secure against a use case in which an attacker tries to distill small portions of the mixture. Step H) allows determining an atom and/or functional group portion value for each atom marker M1 and M2.

Based on the atom and/or functional group portion value determined in step H) for each atom marker M1 and M2, step I) comprises calculating a marker mixture Mₓ composition based on the weight fraction of each atom and/or functional group in each marker M1 and M2 and the purity of markers M1 and M2. As shown in table 4, the marker concentration R4 of marker E1-M1 in the marker mixture Mₓ for a 10 ppm overall atom and/or functional group concentration can be calculated as R4=R1*(100/R2) *(100/R3), wherein R1 is the ratio of the atom and/or functional group for a 10 ppm atom concentration of marker E1-M1, R2 is the weight ratio of the atom and/or functional group in the marker E1-M1 and R3 is the purity of the marker E1-M1.

The final step J) comprises preparing the marker mixture Mₓ by mixing the marker amounts determined in step I) and adding it to the hydrocarbon product HP at about the Cₜ-value.

The HP may be marked by a manual or automatic marking process. Manual marking of petroleum hydrocarbons is conventionally achieved by dispensing a marker solution into petroleum hydrocarbon contained within a petroleum hydrocarbon compartment of petroleum hydrocarbon transportation vehicles (for example trucks).

The method of marking described herein provides, among others, the advantage that the presence of the markers containing the at least one same atom and/or functional group does not alter the distillation profile of the marked HP. Furthermore, each marker distills independently from the other according to its own distillation behavior. Thus, the distillation profile of the weighted mix of the at least two markers in the marked HP is equivalent to the corresponding weighted sum of the (isolated) marker distillation profile in the marked HP. The method described herein offers the advantage that the proportion of the at least two markers containing the at least one same atom and/or functional group is such that the distillation profile of the detected and analyzed atom and/or functional group substantially matches that of the marked HP. Thus, if 50% of the marked HP is distilled, about 50% of the detected and analyzed atom and/or functional group is distilled as well. This allows not only qualitative but also quantitative evaluation of the marked HP.

Another embodiment is directed to a method of detecting adulteration in a hydrocardon product (HP) comprising the steps of:
a) sampling a HP marked according to the method described herein, said HP having a predetermined C-value;
b) detecting and calculating the Cₜ-value in the sampled HP by a suitable analytical technique;
c) assessing the potential adulteration of the HP by comparing the Cₜ-value determined in step b) with the pre-determined Cₜ-value of step a).

The marked HP can be sampled at any suitable sampling point in the HP supply chain e.g. at the service station or storage place. While not necessary, though preferably, the amount of the at least one same atom and/or functional group is detected and analyzed by the same analytical technique as used in the method of marking the HP described herein. The calculated amount in the sampled HP is compared against the pre-determined Cₜ-value i.e. the concentration at which the HP was initially marked before entering the supply chain. A significant reduction in the amount of the determined Cₜ-value might indicate that the marked HP has been adulterated somewhere along the supply chain.

Another embodiment is directed to a method of detecting adulteration in a hydrocarbon product (HP) comprising the steps of:
a) sampling a first HP;
b) detecting and, if present, quantifying an atom and/or functional group by a suitable analytical technique;
c) determining the amount of a second HP marked according to the method described herein, in the first HP, based on the quantified atom and/or functional group from step b).

In this embodiment, it is expected that the first HP sampled at the sampling point is unmarked. However, in case an atom and/or functional group is detected by a suitable analytical technique, such as those described herein, this indicates that a second HP marked preferably according to the method described herein has been added to the first HP resulting in adulteration. In such a case, the detected atom and/or functional group may be quantified and the quantified amount of the atom and/or functional group directly correlates to the amount of the second HP, marked according to the method described herein, which has been mixed or added to the first HP.

The skilled person can envisage several modifications to the specific embodiments described above without departing from the spirit of the present invention. Such modifications are encompassed within the present invention.

Further, any documents referred to throughout this specification are hereby incorporated by reference in their entirety as set forth in full herein.

### EXAMPLES

The present invention is now described in more details with reference to non-limiting examples. The examples below provide more details for the method, in particular for the calculation of the composition of a marker mixture and the method for marking a hydrocarbon product with the marker mixture.

### Determination of the Distillation Curve of the Neat Hydrocarbon Product HP

Three hydrocarbon products HP1-3 were selected: SOCAR 95 Suisse gasoline (E1) and SOCAR Suisse diesel (E2) both from SOCAR Energy Switzerland, and kerosene jet A-1 from Total Energies (E3).

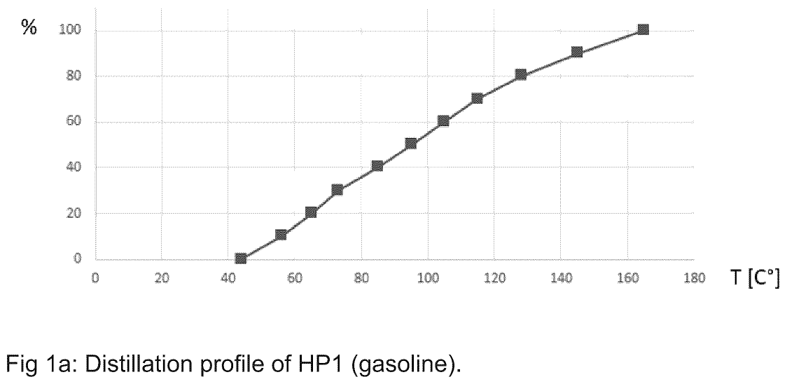

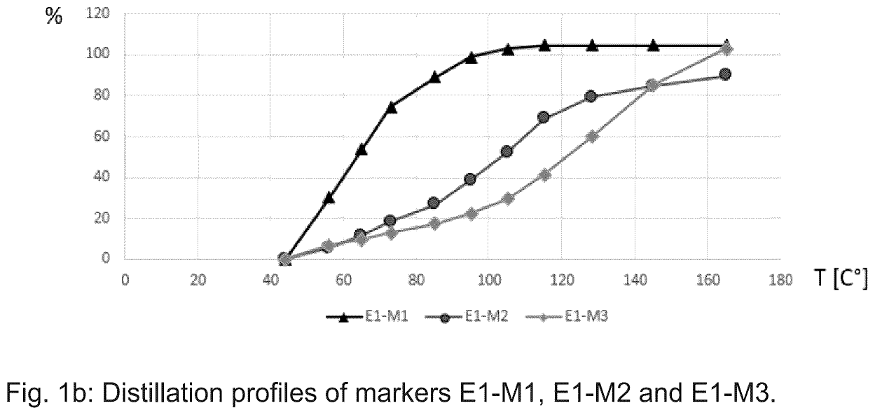

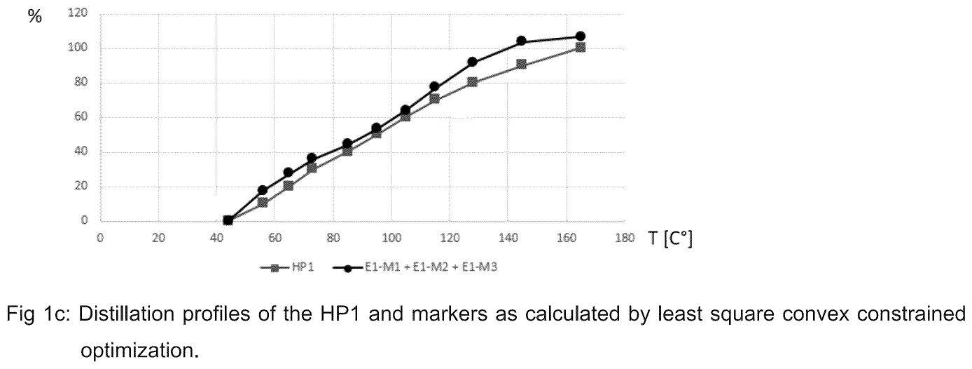

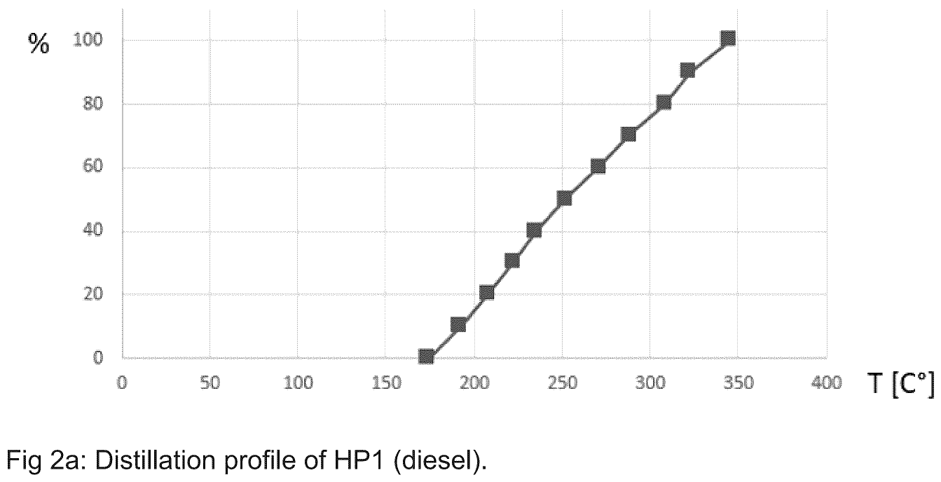

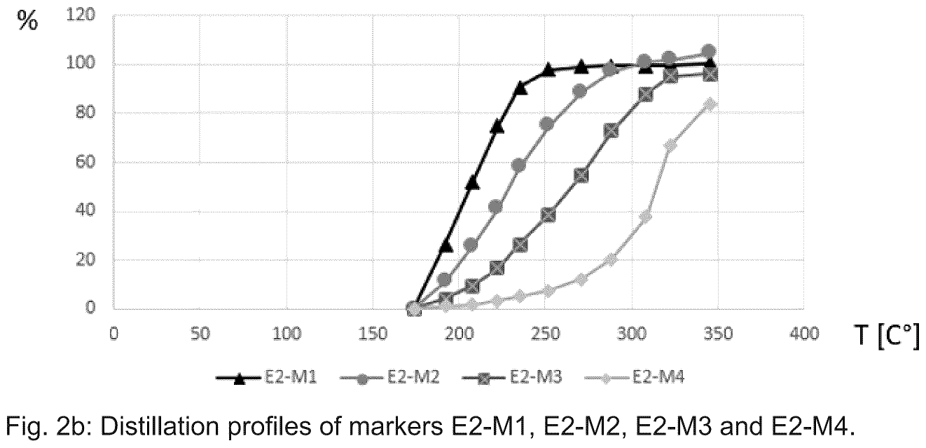

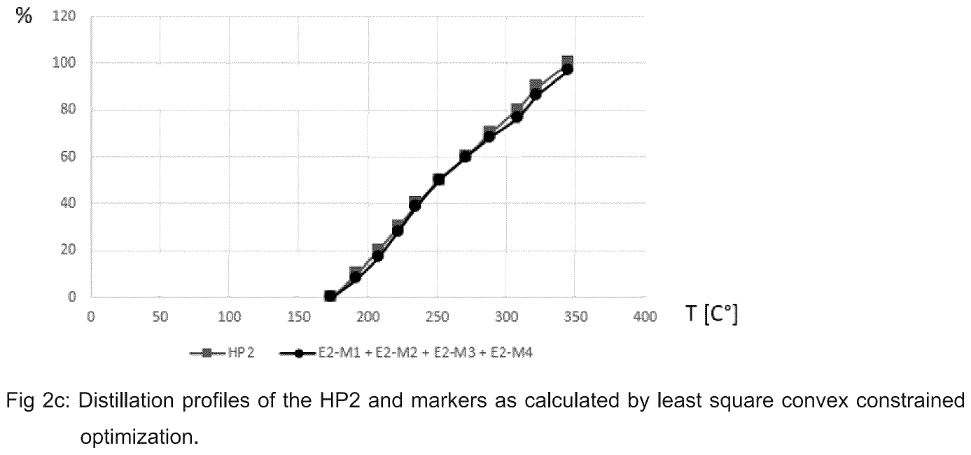

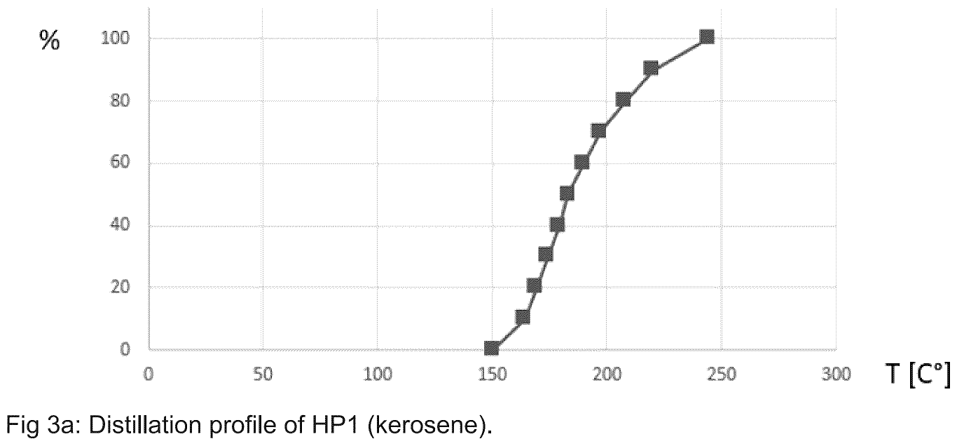

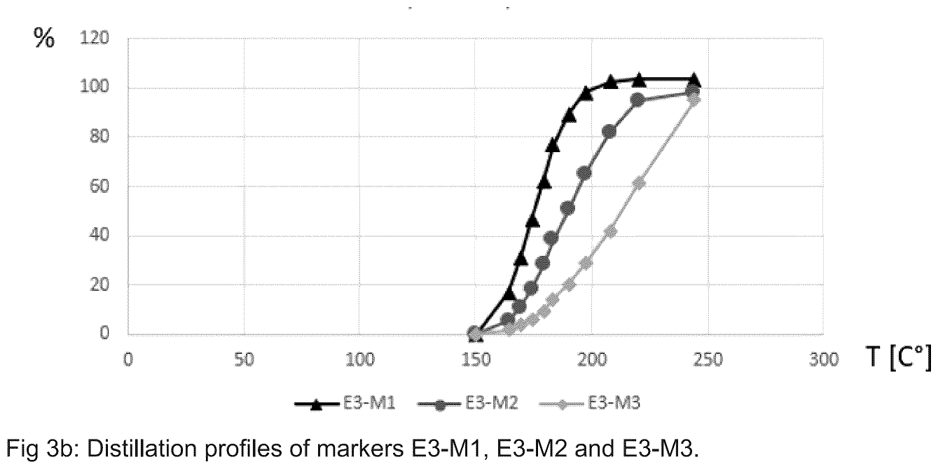

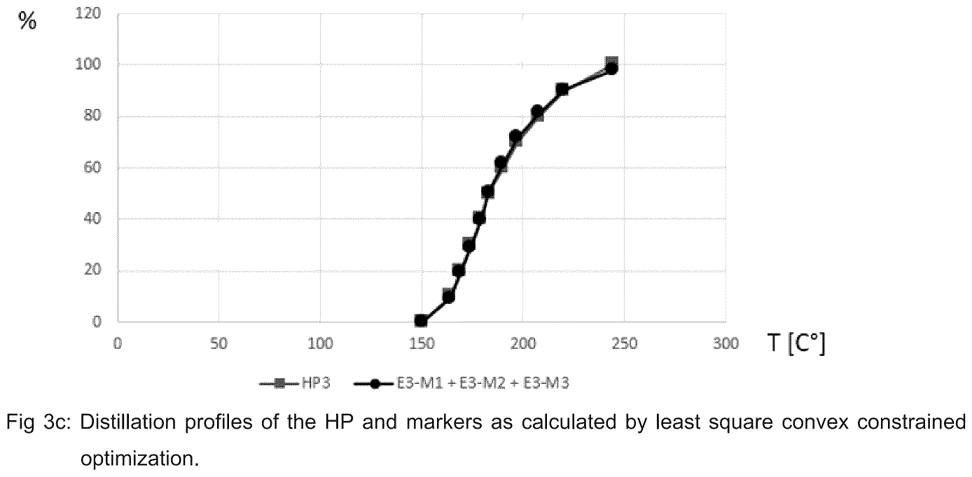

A sample of 100 ml of each hydrocarbon product was distilled independently according to the ASTM D-86 norm. The temperature of the distillation start T(BpL) was recorded. Fractions of 10 ml were collected and the temperature ranges at which each fraction was collected were recorded, the temperature of the last fraction collection was the temperature T(BpE). The obtained distillation profiles for each neat hydrocarbon product HP1-3 are shown in FIGS. 1a, 2a and 3a, respectively.

The distillation temperatures range of each hydrocarbon product HP are shown in table 1:

TABLE 1
Hydrocarbon products to be marked and their boiling point range

| | Hydrocarbon | T(BpL) | T(BpE) |
|---|---|---|---|
| Ex. | Product (HP) | [° C.] | [° C.] |
| E1 | Gasoline (HP1) | 44 | 165 |
| E2 | Diesel (HP2) | 174 | 340 |
| E3 | Kerosene (HP3) | 135 | 244 |

### Selection of the Markers of Example E1-E3

The markers for the examples E1-E3 were selected as disclosed in table 2:

TABLE 2
List of markers used in examples E1-E3

| | | | | Ratio | | |
|---|---|---|---|---|---|---|
| | | | MW | AM/MW³⁾ | | |
| Marker | IUPAC Name | CAS Nr. | [g/mol] | [%] | Bp [° C.] ¹⁾ | Purity |
| E1-M1 | 2-Chloropropane | 75-29-6 | 78.54 | 45.1 | 35.7 | TCI, >99% |
| E1-M2 | 1-Chloropentane | 543-59-9 | 106.59 | 33.2 | 107.8 | Thermo Scientific, |
| | | | | | | 98% |
| E1-M3 | Chlorohexane | 544-10-5 | 120.62 | 29.4 | 135 | Fluka, 99% |
| E2-M1 | Iodobenzene | 591-50-4 | 204.01 | 62.2 | 188.4 | Sigma Aldrich, |
| | | | | | | 99% |
| E2-M2 | 1,4-Diiodobutane | 628-21-7 | 309.92 | 81.9 | 246° C. ²⁾ | Sigma Aldrich, |
| | | | | | | 98% |
| E2-M3 | 1,6-Diiodohexane | 629-09-4 | 337.97 | 75.1 | 307 ²⁾ | Sigma Aldrich, |
| | | | | | | 98% |
| E2-M4 | 1- | 544-77-4 | 352.34 | 36.0 | 357 ²⁾ | Sigma Aldrich, |
| | Iodohexadecane | | | | | 95% |
| E3-M1 | Octamethylcyclo- | 556-67-2 | 296.62 | 37.9 | 175 | Sigma-Aldrich, |
| | tetrasiloxane | | | | | 98% |
| E3-M2 | Decamethylcyclopentasiloxane | 541-02-6 | 370.77 | 37.9 | 210 | Acros, 97% |
| E3-M3 | Dodecamethylcyclohexasiloxane | 540-97-6 | 444.92 | 37.9 | 245 | TCI, >97% |

¹⁾ Markers boiling point temperatures according to SciFinder ®
²⁾ Markers boiling point temperatures according to SDS
³⁾Weight percentage of the atom relative to the molecular weight of each marker

### Example E1

### Determination of the Marker Mixture Composition

Three solutions (100 ml) were independently produced by individually mixing the markers E1-M1, E1-M2 and E1-M3 in gasoline HP1 in a volumetric flask and stirring the three solutions by hand for 1 minute at room temperature (step D). The amount of the markers was selected so that the concentration of atom was in the range of the targeted total concentration Cₜ (e.g. for example E1, Cₜ was about 10 ppm). The exact concentration of the CI-atom was determined by XRF-analysis and was about 10 ppm (10 mg per liter) in each of the three solutions (step D) (see table 3).

A 100 ml sample of each of the three solutions was individually distilled and 10 ml fractions were collected (step E).

The concentration of CI-atom in each fraction was determined by XRF-analysis and the CI-atom mass content of each fraction, as well as the Cl-atom mass amount percentage of each fraction relative to the total CI-atom mass in the initial 100 ml sample and the cumulative atom mass amount percentages of distilled CI-atom were calculated (see table 3 e.g. for the solution of E1-M3 in HP1; the solutions of E1-M1 and E1-M2 in HP1 are measured and calculated analogously). For each of the three solutions, the cumulative atom mass amount percentages of distilled CI-atom were reported against the distillation temperature (FIG. 1b).

For the analysis a SPECTROCUBE ED-XRF(SPECTRO Analytical Instruments Gmbh., Kleve, Germany) was used. The analysis conditions were selected for heavy elements (3 keV<E<6 keV), and a tube voltage of 22.5 kV with a 1 mA was set with current regulation setting ON. Analysis time was set to 300 seconds. Each element was calibrated with a 7-point calibration curve, and the Extended Compton mathematical model was selected with a logarithmic fitting.

TABLE 3

| | | Distillation | | | | Cumulative |
|---|---|---|---|---|---|---|
| | Cumulative | temperature | | | Atom mass | atom mass |
| Fraction | distilled | range of the | Atom | Atom | amount | amount |
| Nr. | volume | fraction | concentration | mass | percentage | percentage |
| F1-X | (ml) | (° C.) | (ppm) | (mg) | (%)¹⁾ | (%)²⁾ |
| 0 | 0 | — | 11.1 | 1.11 | | |
| 1-1 | 10 | 44-56 | 7.3 | 0.073 | 6.58 | 6.58 |
| 1-2 | 20 | 56-65 | 3.1 | 0.031 | 2.79 | 9.37 |
| 1-3 | 30 | 65-73 | 3.6 | 0.036 | 3.24 | 12.61 |
| 1-4 | 40 | 73-85 | 5 | 0.05 | 4.50 | 17.11 |
| 1-5 | 50 | 85-95 | 5.8 | 0.058 | 5.22 | 22.33 |
| 1-6 | 60 | 95-105 | 8 | 0.08 | 7.21 | 29.54 |
| 1-7 | 70 | 105-115 | 12.8 | 0.128 | 11.53 | 41.07 |
| 1-8 | 80 | 115-128 | 20.9 | 0.209 | 18.82 | 59.89 |
| 1-9 | 90 | 128-145 | 28.3 | 0.283 | 25.49 | 85.38 |
| 1-10 | 100 | 145-165 | 19.6 | 0.196 | 17.65 | 103.03 ⁴⁾ |

¹⁾E.g. for calculating atom mass amount percentage for fraction F1-1: 100/1.11 * 0.073 = 6.58%.
²⁾E.g. for calculating cumulative atom mass amount percentage for fraction F1-2: 6.58 + 2.79 = 9.37%

The optimum marker (E1-M1, E1-M2 and E1-M3) mixture composition was calculated, based on the three distillation profiles, by least square convex constrained optimization as an example for a regression analysis, such that the distillation profile of the marker mixture M1 (based on the atom contribution from each marker) substantially overlaps with the distillation profile of the neat hydrocarbon product HP1 (FIG. 1c) (step H). The resulting wt. % of atom and/or functional group from each marker E1-M1, E1-M2 and E1-M3 in the marker mixture M1 obtained from the least square convex constrained optimization are reported in table 4. The derived marker composition was calculated, taking into consideration the ratio of the atomic mass of CI relative to the molecular weight of each marker E1-M1, E1-M2 and E1-M3 and their respective purity (table 4).

TABLE 4

| | | Ratio of atom | | | Marker | |
|---|---|---|---|---|---|---|
| | | for a 10 ppm | | | concentration | |
| | | atom | | | for a 10 ppm | Marker |
| | | concentration | Ratio | | atom | percentage |
| | | Cₜ ²⁾ | AM/MW | Purity | concentration | in markers |
| | Ratio of | (R1) | (R2) | (R3) | Cₜ ³⁾ | mixture |
| Marker | atom ¹⁾ ^{[%]} | [ppm] | [%] | [%] | (R4) [ppm] | [wt %] |
| E1-M1 | 22.7 | 2.27 | 45.1 | 99 | 5.08 | 16.9 |
| E1-M2 | 46.8 | 4.68 | 33.2 | 98 | 14.38 | 47.9 |
| E1-M3 | 30.5 | 3.05 | 29.4 | 98 | 10.58 | 35.2 |
| Total | 100 | 10 | | | 30.04 | 100 |

¹⁾ Ratio (%) of atom from each marker in the marker mixture as calculated by least square convex constrained optimization.
²⁾ Concentration ratio of atom from each marker in the marker mixture for a 10 ppm concentration of markers mixture.
³⁾ Ratio of marker in the marker mixture, after correction based on the weight percentage of the atom relative to the molecular weight of each marker (Table 2) and on the purity of each marker (Table 2). Calculated as: R4 = R1 *(100/R2) * (100/R3).

The examples E2 and E3 were calculated in a similar manner. The results are shown in tables 5 and 6.

TABLE 5
Example E2 (hydrocarbon product HP2)

| | | Ratio of atom | | | Marker | |
|---|---|---|---|---|---|---|
| | | for a 10 ppm | | | concentration | |
| | | atom | | | for a 10 ppm | Marker |
| | | concentration | Ratio | | atom | percentage |
| | | Cₜ | AM/MW | Purity | concentration | in markers |
| | Ratio of | (R1) | (R2) | (R3) | Cₜ (R4) | mixture |
| Marker | atom ^{[%]} | [ppm] | [%] | [%] | [ppm] | [wt %] |
| E2-M1 | 12.2 | 1.22 | 62.2 | 99 | 1.96 | 10.46 |
| E2-M2 | 39.6 | 3.96 | 81.9 | 98 | 4.93 | 26.29 |
| E2-M3 | 13.7 | 1.37 | 75.1 | 98 | 1.86 | 9.92 |
| E2-M4 | 34.5 | 3.45 | 36.3 | 95 | 10.00 | 53.33 |
| Total | 100 | 10 | | | | 100 |

TABLE 6
Example E3 (hydrocarbon product HP3)

| | | Ratio of atom | | | Marker | |
|---|---|---|---|---|---|---|
| | | for a 1000 | | | concentration | |
| | | ppm atom | | | for a 1000 | Marker |
| | | concentration | Ratio | | ppm atom | percentage |
| | | Cₜ | AM/MW | Purity | concentration | in markers |
| | Ratio of | (R1) | (R2) | (R3) | Cₜ (R4) | mixture |
| Marker | atom ^{[%]} | [ppm] | [%] | [%] | [ppm] | [wt %] |
| E3-M1 | 47.3 | 473 | 37.9 | 98 | 1273.49 | 47.04 |
| E3-M2 | 24.4 | 244 | 37.9 | 97 | 663.71 | 24.52 |
| E3-M3 | 28.3 | 283 | 37.9 | 97 | 769.80 | 28.44 |
| Total | 100 | 1000⁴⁾ | | | | 100 |

⁴⁾For E3, the Cₜ of Si was selected as 1000 ppm, due to the (lower) sensitivity of XRF for Si (as compared to the XRF sensitivity for halogens).

1. A method for marking a hydrocarbon product (HP) comprising the steps of:
A. selecting a HP to be marked and recording the distillation profile of HP to determine the starting temperature (TBpL) and the ending temperature (TBpE) at which distillation occurs;
B. selecting at least two compatible and distillable markers (M1, M2) for marking the HP, said markers comprising at least one same atom and/or functional group to be analyzed by a suitable analytical technique, wherein M1 and M2 individually have different boiling points and/or different distillation profiles, wherein one of M1 or M2 has a boiling point temperature T(BpM1) being lower than T (BpL) or one of M1 or M2 has a boiling point temperature being within the distillation temperature range of HP [T(BpL)<T(BpM1)<T(BpE)] and being at most 20% higher than T(BpL),
and
wherein one of M1 or M2 has a boiling point temperature T(BpM2) higher than T (BpE) or the other of M1 or M2 has a boiling point temperature being within the distillation temperature range of HP [T(BpL)<T(BpM2)<T(BpE)], and being at most 20% lower than T(BpE);
C. selecting a total atom concentration Cₜ-value, wherein Cₜ-value is selected based on the analytical technique and the at least one same atom and/or functional group to be analyzed by said analytical technique, selected in step B);
D. preparing individual solutions n₁ and n₂ of markers M1 and M2 in the HP, respectively and determining before fractional distillation the initial concentration and the initial total mass of the at least one same atom and/or functional group in n₁ and n₂ by the analytical technique selected in step B);
E. performing separately fractional distillation of the solutions n₁ and n₂ to obtain fractions F;
F. determining the concentration and the total mass of the at least one same atom and/or functional group in each fraction F by the analytical technique selected in step B);
G. calculating the atom mass amount percentage (%) relative to the initial total mass of the at least one same atom and/or functional group determined in step D) in each fraction F and determining the cumulative atom mass amount percentage for each fraction F;
H. determining an atom portion originating from individual markers M1 and M2 in a marker mixture Mₓ by performing a regression analysis to determine a relationship between the cumulative atom mass amount percentage for each fraction F as determined in step G) for each of the solutions n₁ and n₂ and the distillation profile of HP determined in step A) for at least part of the distillation temperature range of HP;
I. calculating a marker mixture Mₓ composition based on the atom portion determined in step H) for each marker M1 and M2, the weight fraction of each atom and/or functional group in each marker M1 and M2 and the purity of markers M1 and M2;
J. preparing the marker mixture Mₓ by mixing the marker amounts determined in step I) and adding it to the hydrocarbon product HP at about the Cₜ-value.

2. The method for marking a hydrocarbon product (HP) according to claim 1, wherein one of M1 or M2 has a boiling point temperature T(BpM1) being lower than T (BpL) or one of M1 or M2 has a boiling point temperature being within the distillation temperature range of HP [T(BpL)<T(BpM1)<T(BpE)] and being at most 15% higher than T(BpL),
and
wherein one of M1 or M2 has a boiling point temperature T(BpM2) higher than T(BpE) or the other of M1 or M2 has a boiling point temperature being within the distillation temperature range of HP [T(BpL)<T(BpM2)<T(BpE)], and being at most 15% lower than T(BpE).

3. The method for marking a hydrocarbon product (HP) according to claim 1, wherein at least four markers M1-M4 are selected in step B).

4. The method for marking a hydrocarbon product (HP) according to claim 1, wherein “i” markers are selected in step B) and wherein i>4.

5. The method for marking a hydrocarbon product (HP) according to claim 1, wherein the at least two markers M1 and M2 comprise an atom.

6. The method for marking a hydrocarbon product (HP) according to claim 1, wherein the at least two markers M1 and M2 comprise a halogen atom.

7. The method for marking a hydrocarbon product (HP) according to claim 1, wherein the analytical technique selected in step B) is based on X-ray fluorescence.

8. The method for marking a hydrocarbon product (HP) according to claim 1, wherein the Cₜ-value in step C) is between 10-2000 ppm.

9. The method according to claim 1, wherein the hydrocarbon product (HP) is a fuel, preferably diesel, kerosene or gasoline.

10. A method of detecting adulteration in a hydrocardon product (HP) comprising the steps of:
a) sampling a HP marked according to claim 1, said HP having a predetermined Cₜ-value;
b) detecting and calculating the Cₜ-value in the sampled HP by a suitable analytical technique;
c) assessing the potential adulteration of the fuel by comparing the Cₜ-value determined in step b) with the pre-determined Cₜ-value of step a).

11. A method of detecting adulteration in a hydrocarbon product (HP) comprising the steps of:
a) sampling a first HP;
b) detecting and, if present, quantifying an atom and/or functional group by a suitable analytical technique;
c) determining the amount of a second HP marked according to claim 1 in the first HP, based on the quantified atom and/or functional group from step b).

12. (canceled)

13. The method for marking a hydrocarbon product (HP) according to claim 1, wherein individual solutions n₁ and n₂ of markers M1 and M2 in the HP, respectively, are prepared at the Cₜ-value.

14. The method for marking a hydrocarbon product (HP) according to claim 8, wherein the Cₜ-value in step C) is between 10-1000 ppm.

15. The method according to claim 9, wherein the fuel is diesel, kerosene or gasoline.
